(19) United States
(12) Patent Application Publication
(10) Pub. No.: US 2011/0245693 A1
(43) Pub. Date: Oct. 6, 2011

(54) INTRAVASCULAR PRESSURE SENSING

(75) Inventors: Roger N. Hastings, Maple Grove, MN (US); Leonard B. Richardson, Brooklyn Park, MN (US); Kevin D. Edmunds, Ham Lake, MN (US); Michael J. Pikus, Golden Valley, MN (US)

(73) Assignee: Boston Scientific Scimed, Inc., Maple Grove, MN (US)

(21) Appl. No.: 13/073,687

(22) Filed: Mar. 28, 2011

Related U.S. Application Data
(60) Provisional application No. 61/319,071, filed on Mar. 30, 2010.

(51) Int. Cl. A61B 5/0215

(52) U.S. Cl. 600/486

(57) ABSTRACT

Devices, systems, and methods associated with pressure sensing are described herein. In one or more embodiments, an intravascular pressure sensing device includes a magnetic sensing element fixedly positioned within a sensor tube, a magnet located a distance from the magnetic sensing element within the sensor tube, the magnet movably positioned within the sensor tube via a ferrofluid magnetically attached to the magnet, and an amount of compressible fluid sealed between the magnetic sensing element and the magnet.

### PRIORITY INFORMATION

This application claims priority to U.S. Provisional Application No. 61/319,071 filed on Mar. 30, 2010, the specification of which is incorporated herein by reference.

### TECHNICAL FIELD

The present disclosure relates generally to pressure sensing devices, systems, and methods, and more particularly, to intravascular pressure sensing devices, systems, and methods.

### BACKGROUND

Pressure sensors can be used in interventional medicine to provide feedback on the status of medical procedures as they are being performed and to monitor the effectiveness of a medical procedure after completion. Pressure sensors can be used in and/or with medical devices that are used to perform medical procedures within blood vessels. The size of some blood vessels can restrict the size of pressure sensors that can be used with medical devices in blood vessels. Medical procedures using pressure sensors in blood vessels can be exposed to conditions with variance in pressure and temperature. A pressure sensor that can remain operational when exposed to variance in pressure and temperature can be used in and/or with a workhorse medical device, such as a guidewire.

There are a number of pressure sensing techniques that can be used to sense blood pressure. Microelectromechanical systems (MEMs) sensors and/or inductive pressure sensors, among other types of sensor technologies, can be used with and/or integrated into medical devices to sense blood pressure. These medical devices used during medical procedures that are performed at least partially within a blood vessel are small enough to be placed and maneuvered through a blood vessel while maintaining their functionality.

Pressure sensors can be used to measure blood pressure at a number of locations. A medical device including a single pressure sensor can be moved to a number of locations to determine blood pressure at various locations, for example distal and proximal to a lesion. Also, a medical device including two or more pressure sensors can be used to determine blood pressure at the location of each of the pressure sensors.

A pressure sensor that is sized to be used in blood vessels for medical procedures, that can measure pressure changes with medically relevant resolutions, and that can withstand the environmental challenges of a blood vessel is desired.

### BRIEF DESCRIPTION OF THE DRAWINGS

FIG. 1A illustrates a longitudinal cross sectional view of an intravascular magnetic pressure sensing device in accordance with one or more embodiments of the present disclosure.

FIG. 1B illustrates a transverse cross sectional view of a portion of the intravascular magnetic pressure sensing device shown in FIG. 1A.

FIG. 1C illustrates a transverse cross sectional view of a portion of the intravascular magnetic pressure sensing device shown in FIG. 1A.

FIG. 2A is a graph illustrating sensor magnetization versus applied magnetic field associated with an intravascular magnetic pressure sensing device in accordance with one or more embodiments of the present disclosure.

FIG. 2B is a graph illustrating magnetic permeability versus applied magnetic field associated with an intravascular magnetic pressure sensing device in accordance with one or more embodiments of the present disclosure.

FIG. 3 illustrates a longitudinal cross sectional view of an intravascular magnetic pressure sensing device integrated into a guidewire in accordance with one or more embodiments of the present disclosure.

FIG. 4 illustrates an intravascular pressure sensing system including a first and a second sensing device integrated in a guidewire in accordance with one or more embodiments of the present disclosure

FIG. 5 illustrates a longitudinal cross sectional view of an intravascular magnetic pressure sensing device in accordance with one or more embodiments of the present disclosure.

FIG. 6 is a block diagram of an intravascular pressure sensing system in accordance with one or more embodiments of the present disclosure.

### DETAILED DESCRIPTION

Devices, systems, and methods associated with pressure sensing are described herein. In one or more embodiments, an intravascular pressure sensing device includes a magnetic sensing element fixedly positioned within a sensor tube, a magnet located a distance from the magnetic sensing element within the sensor tube, the magnet movably positioned within the sensor tube via a ferrofluid magnetically attached to the magnet, and an amount of compressible fluid sealed between the magnetic sensing element and the magnet.

In the following detailed description of the present disclosure, reference is made to the accompanying drawings that form a part hereof, and in which is shown by way of illustration how one or more embodiments of the disclosure may be practiced. These embodiments are described in sufficient detail to enable those of ordinary skill in the art to practice the embodiments of this disclosure, and it is to be understood that other embodiments may be utilized and that process, electrical, and/or structural changes may be made without departing from the scope of the present disclosure.

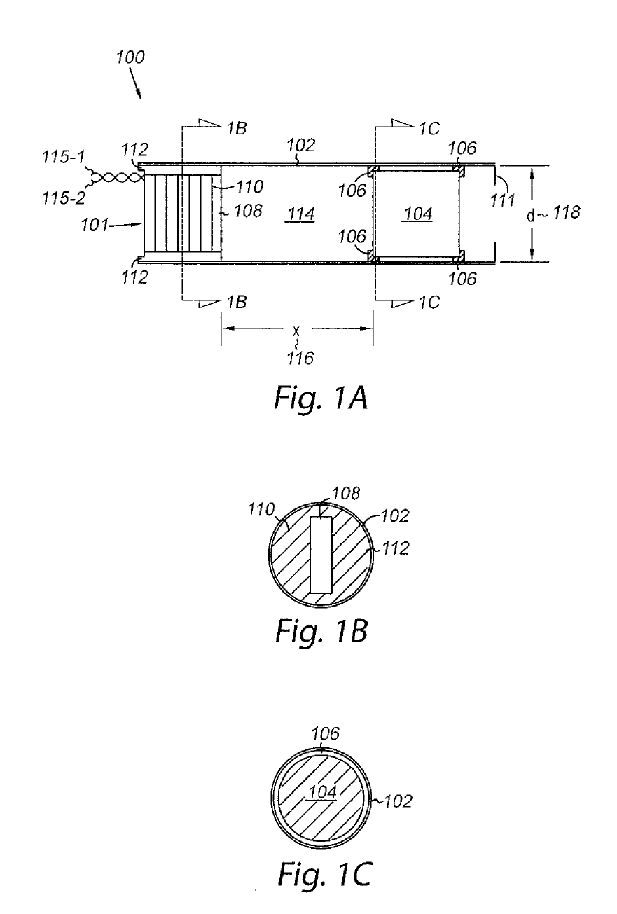

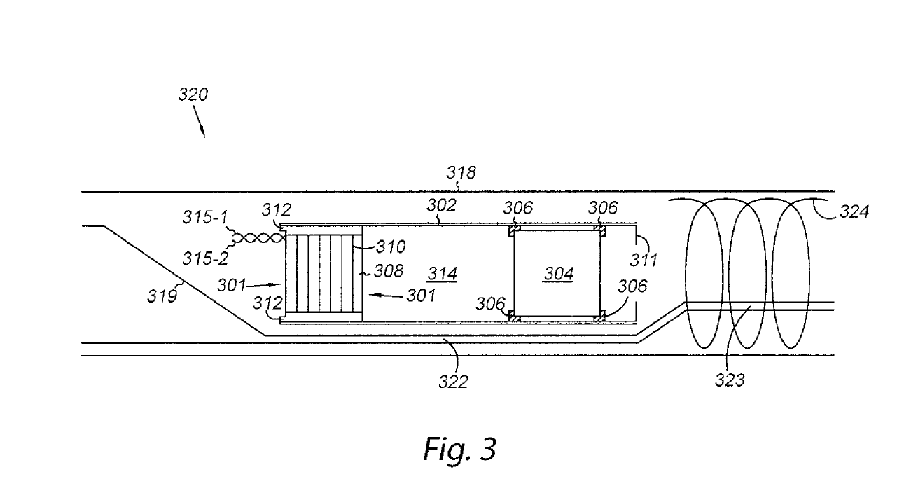

The figures herein follow a numbering convention in which the first digit or digits correspond to the drawing figure number and the remaining digits identify an element or component in the drawing. Similar elements or components between different figures may be identified by the use of similar digits. For example, 110 may reference element “10” in FIG. 1A, and a similar element may be referenced as 310 in FIG. 3. As will be appreciated, elements shown in the various embodiments herein can be added, exchanged, and/or eliminated so as to provide a number of additional embodiments of the present disclosure. In addition, the proportion and the relative scale of the elements provided in the figures are intended to illustrate various embodiments of the present invention and are not to be used in a limiting sense.

FIG. 1A illustrates a longitudinal cross sectional view of an intravascular magnetic pressure sensing device 100 in accordance with one or more embodiments of the present disclosure. FIGS. 1B and 1C illustrate transverse cross sectional views of portions of the intravascular magnetic pressure sensing device 100 shown in FIG. 1A.

In various embodiments, and as illustrated in FIG. 1A, pressure sensing device 100 includes a magnetic sensing element 101 and a magnet 104 spaced a predetermined distance (“x”) 116 apart within a sensor tube 102. The sensor tube 102 can be a non-metallic tube made of polyimide and can have a length of about 0.040 inches (1.016 millimeters (mm)), an outer wall diameter (“d”) 118 of about 0.010 inches (0.254 mm), and a wall thickness of about 0.0005 inches (0.0127 mm), for example. Other dimensions of the sensor tube 102 are possible. In some embodiments, the sensor tube 102 can be a non-magnetic tube made of 316 series stainless steel, MP35N, nitinol, or other non-magnetic metal alloy having a length of approximately 0.040 inches, outside diameter of approximately 0.014 inches, and wall thickness of approximately 0.002 inches.

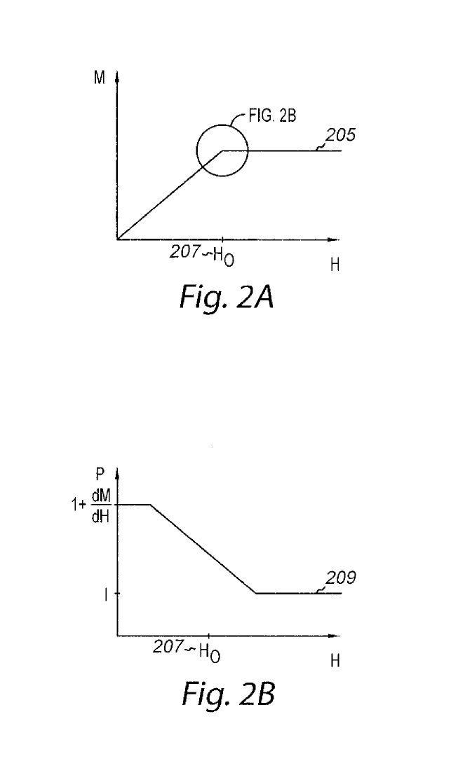

The magnet 104 can be cylindrical and can have a diameter that is approximately 0.001 inches (0.0254 mm) smaller than the diameter 118 of the sensor tube 102. As discussed herein, in various embodiments, the magnet 104 is placed at a predetermined distance 116 from the magnetic sensing element 101. As described in connection with FIGS. 2 and 3, the predetermined distance 116 can be determined based on the linear dependence of the permeability of the material 108 under an applied magnetic field.

The magnetic sensing element 101 of sensing device 100 can be a magnetic sensor such as a Hall effect sensor, giant magneto-resistive (GMR) sensor, or saturable core sensor, among other types of magnetic sensors. In various embodiments, the magnetic sensing element 101 is fixedly secured within the sensor tube 102 and used to sense changes in inductance of coil 110 in response to movement of magnet 104 relative to sensing element 101. That is, sensing element 101 can be used to sense inductance changes in response to changes in the distance 116 (e.g., due to blood pressure changes within a body lumen).

In the embodiment illustrated in FIG. 1A, the sensing element 101 is a saturable core sensor 101 (e.g., a saturable core magnetometer). The sensing element 101 can include a wire filament wound around a permeable, non-hysteretic material 108 (e.g., Metglas® available from Metglas®, Inc., Jimmy W. Jordan, 440 Allied Drive, Conway, S.C. 29526 or other permeable material having low or zero associated hysteresis) to form a number of sensor windings 110. The sensor windings 110 may be made of silver or copper wire and the number of windings may vary and may be layered around material 108. The material 108 can be approximately 0.009 inches (0.2286 mm) long, 0.009 inches (0.2286 mm) wide, and 0.001 inches (0.0254 mm) thick, for example. Other dimensions for the material 108 are also possible. In the embodiment illustrated in FIG. 1A, the sensing element 101, which can include the material 108 and windings 110, can be glued to the inner surface of the sensor tube 102, creating a glue seal 112 at the proximal end of the sensor tube 102.

The sensor windings 110 can include a pair of conductive sensing leads 115-1 and 115-2. The leads 115-1 and 115-2 can be electrically coupled to and provide signals to a measurement device (not shown in FIG. 1A), which can be used to determine blood pressure based on the measured inductance of the windings 110. In contrast to some previous intravascular pressure sensing devices, which require three or more leads to provide pressure measurement signals, one or more embodiments of the present disclosure can use only two leads (e.g., 115-1 and 115-2).

As discussed herein, in various embodiments, the magnet 104 is movable within the sensor tube 102. For example, the magnet 104 can be configured to slide longitudinally within the sensor tube 102. In the embodiment illustrated in FIG. 1A, the magnet 104 is movably positioned within the sensor tube 102 via an amount of ferrofluid 106 magnetically attached to the magnet 104. A ferrofluid can be a colloidal suspension of magnetic particles in a carrier liquid. The ferrofluid can be attracted to a magnet and can become coupled to a magnet due to magnetic forces. A ferrofluid can reduce friction between surfaces allowing two surfaces with a ferrofluid between them to move easier relative to each other. As shown in FIG. 1A, the ferrofluid 106 can gather at the regions of highest magnetic field of the magnet 104, and can prevent the magnet 104 from contacting the inner surface of the sensor tube 102. As such, the ferrofluid 106 surrounding the magnet 104 creates a fluid tight ferrofluid seal between an amount of compressible fluid 114 within the sensor tube 102 and the outside of the sensor tube.

The compressible fluid 114 is sealed between the magnetic sensing element 101 and magnet 104 within the sensor tube 102 can be an inert gas 114 such as xenon (Xe) or other gas such as Argon or Krypton that does not diffuse through sensor tube 102 and has low permeability into the ferrofluid 106. In one or more embodiments, after the magnetic sensing element 101 is secured in the sensor tube 102, the sensor tube 102 can be placed into an air tight fixture, such as a glove box. A glove box is a sealed container that can allow a user to be present in one atmosphere while manipulating an object that is in a separate atmosphere, such as a vacuum, for example. The tube 102 is then evacuated of air and back filled with the gas 114. The gas is maintained at standard pressure and temperature during the procedure. As used herein, standard temperature may be defined as body temperature of 310 Kelvin (37° Celsius (C)), and standard pressure may be defined as one atmosphere plus 100 mm Hg (e.g., 860 mm Hg, which is near an average human blood pressure). The tube is filled with the inert gas to a standard pressure. In this example, the magnet 104 and ferrofluid 106 are then introduced into the sensor tube 102 to create the fluid tight ferrofluid seal.

In various embodiments, the sensor tube 102 can include a stop member 111 configured to prevent movement of the magnet 104 out of the sensor tube 102. As shown in FIG. 1A, the stop member 111 can be attached to the inner surface of the sensor tube 102.

FIG. 1B illustrates a transverse cross sectional view of the magnetic sensing element 101 of sensing device 100 shown in FIG. 1A. FIG. 1B illustrates the glue seal 112 between the inner surface of sensor tube 102 and permeable material 108.

FIG. 1C illustrates a transverse cross sectional view of the magnet 104 and ferrofluid 106 of sensing device 100 shown in FIG. 1A. In FIG. 1C, the magnet 104 is surrounded by ferrofluid 106. The ferrofluid 106 levitates the magnet 104 allowing the magnet 104 and the ferrofluid 106 to move longitudinally within sensor tube 102 of magnetic sensing device 100. As described herein, the magnet 104 can move within sensor tube 102 in response to pressure changes (e.g., intravascular blood pressure changes).

As described herein, in various embodiments, a magnetic sensing device such as device 100 can be incorporated into a pressure sensing guidewire. In some embodiments, multiple sensing devices (e.g., a first and second sensing device 100) can be incorporated into a pressure sensing guidewire. Providing two magnetic sensing devices 100 can provide benefits such as allowing for simultaneous measuring of the distal pressure and proximal pressure associated with a coronary artery lesion, for instance.

FIG. 2A is a graph illustrating permeable material 108 magnetization (M) versus applied magnetic field (14) associated with an intravascular magnetic pressure sensing device in accordance with one or more embodiments of the present disclosure. FIG. 2B is a graph illustrating magnetic permeability μ (e.g., μ equals the change in magnetization divided by the change in applied magnetic field plus one (μ=1+dM/dH)) of permeable material 108 versus applied magnetic field (H) associated with magnet 104 in an intravascular magnetic pressure sensing device in accordance with one or more embodiments of the present disclosure.

For FIGS. 2A and 2B, consider a magnetic pressure sensing device (e.g., magnetic pressure sensing device 100 described with respect to FIG. 1A) that is a saturable core magnetometer. In this example, consider a saturable core magnetometer having a core material (e.g., material 108 described with respect to FIG. 1A) with low or zero hysteresis (e.g. a Metglas® core available from Metglas®, Inc., Jimmy W. Jordan, 440 Allied Drive, Conway, S.C. 29526). As illustrated by curve 205 in FIG. 2A, the magnetization (M) of the core material increases linearly and reaches a maximum value due to an applied field, H, at saturation point, H₀, 207. Curve 205 can be retraced as the applied field, H, is reduced to zero (i.e., no hysteresis).

Curve 209, illustrated in FIG. 2B, shows that the permeability of the core material changes rapidly from a maximum to a minimum value (e.g., one) at saturation point 207. As such, a saturable core magnetometer is sensitive to small applied magnetic field variations at or near saturation point, H₀, 207. Therefore, the saturable core can be biased at the saturation point, H₀, 207 in order to provide effective sensitivity.

For instance, the predetermined distance x 116 shown in FIG. 1A can be set such that it corresponds to the saturation point H₀ of the core material 108 of sensing element 101. In operation, intravascular pressure changes can cause movement of magnet 104 within sensor tube 102, which can change the distance x between the sensing element 101 and the magnet 104. The changes in the distance x correspond to changes in the magnetic field applied to the core 108 as magnet 104 moves against fluid column 114 in response to blood pressure changes, resulting in inductance changes in windings 110, which correspond to pressure changes.

The inductance associated with windings 110 can be measured for various distances between the magnet 104 and the sensing element 101, which are then converted to a corresponding pressure in a calibration procedure.

As such, and as described herein, magnetic pressure sensors can be used to measure pressure changes based on the change in inductance of the pressure sensing element. The inductance of a pressure sensing element can change relative to the magnetic field applied to the pressure sensing element. A magnet in a pressure sensor device can be moved, changing its position relative to a sensing element, based on changes in the pressure surrounding the pressure sensing device. The magnet's position relative to a sensing element can determine the magnitude of the applied magnetic field. The change in the pressure surrounding the pressure sensing device can then be calibrated to correspond to the change in inductance of a pressure sensing element, allowing the pressure surrounding the pressure sensing device to be measured by sensing the inductance of the pressure sensing element.

As an example, a measurement device electrically coupled to the sensing device 100 can use a look up table of inductive reactance versus pressure in order to determine the pressure for a given inductance of windings 110. As such, pressure increases and decreases can be measured with equal resolution and scale. In one or more embodiments, the pressure changes can be measured with a resolution of for example, 0.4 mm Hg, or better. For example, a change in blood pressure from 840 mm Hg to 880 mm Hg is roughly a 5 percent change in absolute pressure. Inductance can be measured at a specific frequency and changes in inductance can be measured in a small band pass around this frequency. The 5 percent change in absolute pressure (e.g., 40 mm Hg) can be measured to 1 part in 100, therefore the resolution of the pressure measurements can equal 0.4 mm Hg.

FIG. 3 illustrates a longitudinal cross sectional view of an intravascular magnetic pressure sensing device 300 integrated into a guidewire 320 in accordance with one or more embodiments of the present disclosure. The magnetic pressure sensing device 300 can be a magnetic pressure sensing device such as device 100 described in connection with FIGS. 1A-1C. In the embodiment illustrated in FIG. 3, the guidewire 320 includes an elongate tube 318 (e.g., a hypotube or slotted hypotube) and a tapered core wire 319.

In FIG. 3, the magnetic pressure sensing device 300 is integrated in the guidewire 320 near a coil spring tip 324 of the guidewire (e.g., near a distal end of the guidewire 320). As illustrated in FIG. 3, the core wire 319 can include a flattened portion 322 that passes under sensing device 300 and the distal end of the core wire 319 can be used as a shaping wire 323 at the distal end of the guidewire 320 during intravascular procedures.

In the embodiment illustrated in FIG. 3, the magnetic pressure sensing device 300 can be a saturable core magnetic sensing element 301 having a number of sensor windings 310 around permeable material 308. The magnetic sensing element 301 is fixedly positioned at a proximal end of the sensor tube 302 via glue seal 312. The sensing device 300 includes a movable magnet 304 surrounded by an amount of ferrofluid 306, which creates a ferrofluid seal between the magnet 304 and inner surface of sensor tube 302 while allowing for longitudinal movement of the magnet 304 in response to intravascular pressure changes. As described herein, the space between the magnet 304 and magnetic sensing element 301 can be filled with a compressible fluid 314 (e.g., an inert gas such as Xe) that provides a restoring force on magnet 304 when blood pressure changes.

In the embodiment illustrated in FIG. 3, the magnetic pressure sensing device 300 includes only two conductive leads 315-1 and 315-2. The leads 315-1 and 315-2 may exit through a hollow proximal portion (not shown in FIG. 3) of core wire 319 and are electrically coupled to a measurement device (e.g., measurement device 662 shown in FIG. 6). The leads 315-1 and 315-2 provide signals to the measurement device, which are used to determine intravascular pressure measurements. Providing only two leads 315-1 and 315-2 can provide benefits such as integrating the leads into a guidewire with minimal impact on the mechanical properties of the guidewire. For example, the core wire 319 can double as an electrical lead (for example by connecting lead 315-1 to core wire 319, and electrically insulating core wire 319). Furthermore lead 315-2 may be connected to elongate tube 318, so that elongate tube 318 serves as the second electrical lead, providing that tube 318 is suitably electrically insulated, for example, with a coating such as parylene. In one or more embodiments, a guide wire with two full length electrical leads to sensing device 300 integrated along length the of the guidewire can maintain mechanical performance of the guidewire, such as the flexibility profile along the length of the guidewire, one-to-one torque response of the distal end of the guidewire, and the ability to push through a blood vessel without prolapsing.

In one or more embodiments, a sensor tube may be portion of the elongate tube located just proximal of the spring tip. In this embodiment, a core wire can end proximal of the sensing element, and the wall of the sensor tube is thickened in the region of the sensing element to provide torque transmission to the spring tip and to provide strong and safe coupling of the proximal guidewire to the distal end. Sensor tube may include the outside surface of the guidewire along its length and be bonded to the proximal guidewire tube and spring tip.

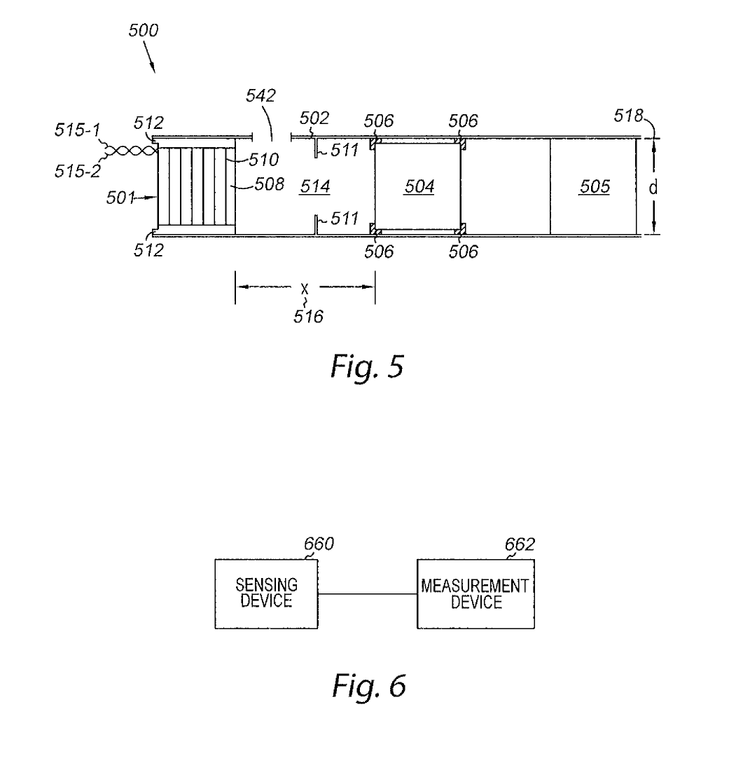

In operation, a guidewire, such as guidewire 320, having a magnetic pressure sensing device 300 incorporated therein, can be used to obtain accurate pressure measurements in a medical procedure. For instance, the guidewire 320 can be traversed through a coronary artery of a patient and the sensor 300 can be positioned proximal to a coronary artery lesion to obtain a proximal pressure measurement and can then be positioned distal to the lesion to obtain a distal pressure measurement. As described below in FIG. 5, in various embodiments, an intravascular pressure sensing system can include two magnetic pressure sensing devices (e.g., magnetic pressure sensing device 300 described with respect to FIG. 3) integrated in a particular guidewire. In such embodiments, the guidewire can be maneuvered such that respective magnetic pressure sensing devices are positioned proximal and distal to the lesion such that proximal and distal pressure measurements can be obtained simultaneously. The proximal and distal pressure measurements associated with a coronary artery lesion may be used to compute the Fractional Flow Reserve (FFR), which is equal to the ratio of blood pressure distal of a lesion to the blood pressure proximal of a lesion. FFR can be computed when a drug is injected that maximally dilates the arterial bed being fed by the lesioned artery. Maximum vasodilation mimics the condition of blood flow during vigorous physical exercise. If the FFR is equal to one, the lesion provides no blockage. If it is equal to zero, the artery is totally occluded. If it is larger than a cut-off value (often defined as 0.8), the lesion is considered insignificant, and need not be treated.

In one or more embodiments, a pressure sensing device can be linearized. For example, a force coil can be wound around a sensor tube near a magnet. Currents in this coil can force the magnet to stay in one position as the pressure around the pressure sensing device changes. The force coil feedback current can then be measured to achieve linearity of the pressure measurement.

In another example, micro-heaters can be installed proximal and distal of the pressure sensing device. The temperature can be raised on one side of the magnet in the pressure sensing device to compensate for a pressure increase on the other side of the magnet. The movement of the magnet would be minimized by offsetting pressure increases with temperature increases, thus linearizing the measurement.

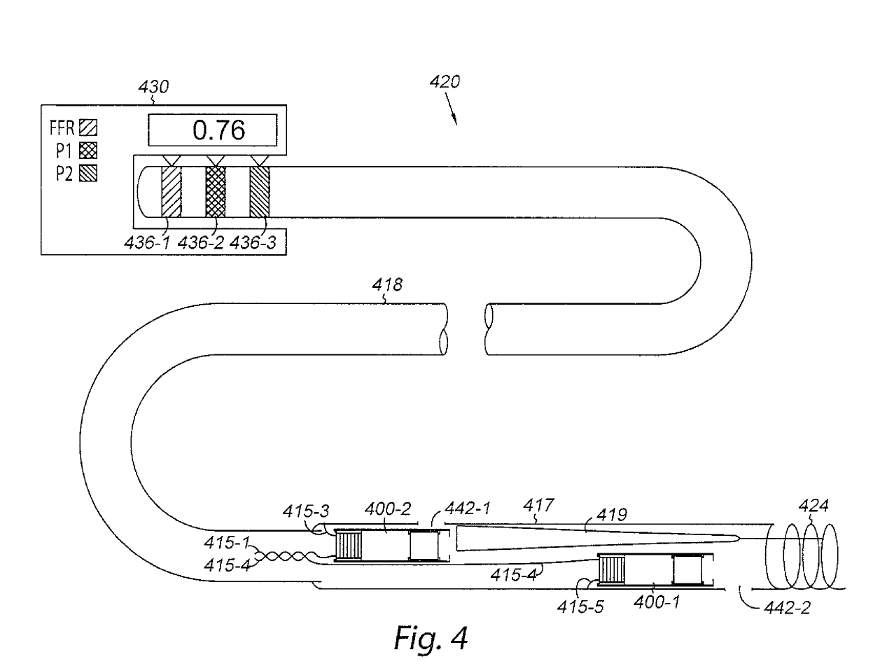

FIG. 4 illustrates an intravascular pressure sensing system including a first and a second sensing device integrated in a guidewire 420 in accordance with one or more embodiments of the present disclosure. In FIG. 4, distal pressure sensing element 400-1 can measure the blood pressure proximal of the spring tip 424, which can be placed under tapered core wire 419 in the distal portion of the elongate tube 417 of guidewire 420 to be distal of a lesion in an artery under investigation. A proximal sensing element 400-2, which is the same as distal pressure sensing element 400-1, can be placed at the transition from the proximal portion of the elongate tube 418 of guidewire 420 to the distal portion of the elongate tube 417 of guidewire 420. The proximal sensing element 400-2 can be located approximately 15 to 25 centimeters (cm) from the distal tip of the guidewire.

The fractional flow reserve (FFR) is defined as the ratio of the distal blood pressure (P1) to proximal blood pressure (P2) during induced hyperemia, e.g. FFR=P1/P2. The proximal blood pressure (P1) can be taken from the patient's arterial fluid line under the assumption that the pressure at the proximal end of the fluid line is equal to the pressure proximal to the lesion. This assumption may be false if the fluid line or guide catheter contains air, if the arterial line pressure sensor is not held at the level of the patient's heart, and/or if the calibration, e.g., volts per mm Hg, is different for the arterial lines senor and the distal guidewire sensing element. Both an offset and a calibration factor mismatch generate errors in the computed FFR.

In FIG. 4, an offset and a calibration factor mismatch can be avoided because the pressure sensors 400-1 and 400-2 are identical with identical calibrations. The outputs of sensors 400-1 and 400-2 are zeroed electronically before the guidewire enters the patient. Thereafter the sensors measure blood pressure with no offset and equal calibration factors in volts per mm Hg.

One of the distal sensor leads 415-5 can be connected to the proximal portion of elongate tube 418, which is electrically insulated from the blood. The second lead 415-4 can travel past sensing element 400-2 along the length of the elongate tube to electrode 436-1 on the proximal shaft of the guidewire. Similarly, lead 415-3 of sensing element 400-2 is connected to the proximal portion of elongate tube 418, sharing a common ground with distal sensing element 400-1. Lead 415-1 from proximal sensing element 400-2 follows sensing lead 415-4 from 400-1 along the length of the elongate tube and terminates in electrode 436-2 on the proximal shaft of the guidewire. Electrode 436-3 on the proximal shaft of the guidewire is connected to the common ground of the proximal portion of elongate tube 418.

In one or more embodiments, a measurement device 462 can be coupled to the proximal portion of elongate tube 418 of the guidewire to make contact with the three electrodes 436-1, 436-2, and 436-3. A digital display on the measurement device 462 can display distal pressure (P1), proximal pressure (P2), and/or FFR=P1/P2. During a procedure, the FFR can be displayed as a vasodilating drug is injected through the guide catheter into the coronary artery under investigation. A sample and hold circuit holds the largest value of the FFR obtained during the injection. This data is also sent by a wireless link to a computer monitor and display unit (not shown). Alternatively, a lead may connect the measurement device 462 to the computer monitor, passing from sterile to non-sterile fluids. Such a lead must be sterilized and carefully passed form the sterile field to the non-sterile area of the operating room.

FIG. 5 illustrates a longitudinal cross sectional view of an intravascular magnetic pressure sensing device in accordance with one or more embodiments of the present disclosure. In various embodiments, and as illustrated in FIG. 5, pressure sensing device 500 includes a magnetic sensing element 501, fixed magnet 505 and a magnet 504 spaced a predetermined distance (“x”) 516 apart within a sensor tube 502. The sensor tube 502 can be similar to the sensor tube (102) discussed above in association with FIGS. 1A-1C.

In the embodiment illustrated in FIG. 5. the sensing element 501 can be similar to the sensing element, e.g., sensing element 101, discussed above in association with FIGS. 1A-1C. In various embodiments, the magnetic sensing element 501 is fixedly secured within the sensor tube 502 by glue seals 512 and is used to sense changes in inductance of coil 510 in response to movement of magnet 504 relative to sensing element 501. That is, sensing element 501 can be used to sense inductance changes in response to changes in the distance 516 (e.g., due to blood pressure changes within a body lumen).

The leads 515-1 and 515-2 can be electrically coupled to and provide signals to a measurement device (not shown in FIG. 5), which can be used to determine blood pressure based on the measured inductance of the windings 510. In contrast to some previous intravascular pressure sensing devices, which require three or more leads to provide pressure measurement signals, one or more embodiments of the present disclosure can use only two leads (e.g., 515-1 and 515-2).

The magnet 504 can be cylindrical and can have a diameter that is approximately 0.001 inches (0.0254 mm) smaller than the diameter 518 of the sensor tube 502. As discussed herein, in various embodiments, the magnet 504 is placed at a predetermined distance 516 from the magnetic sensing element 501. As described in connection with FIGS. 2 and 3, the predetermined distance 516 can be determined based on the linear dependence of the permeability of the material 508 under an applied magnetic field.

As discussed herein, in various embodiments, the magnet 504 is movable within the sensor tube 502. For example, the magnet 504 can be configured to slide longitudinally within the sensor tube 502. In the embodiment illustrated in FIG. 5, the magnet 504 is movably positioned within the sensor tube 502 via an amount of ferrofluid 506 magnetically attached to the magnet 504. As shown in FIG. 5, the ferrofluid 506 can gather at the regions of highest magnetic field of the magnet 504, and can prevent the magnet 504 from contacting the inner surface of the sensor tube 502. As such, the ferrofluid 506 surrounding the magnet 504 creates a fluid tight ferrofluid seal between the sensor tube 102 and the outside of the sensor tube.

In various embodiments, the sensor tube 502 can include a stop members 511 configured to prevent the magnet 504 from contacting blood opening 542 and/or sensing element 501. As shown in FIG. 5, the stop members 511 can be attached to the inner surface of the sensor tube 502.

In FIG. 5, as blood enters the pressure sensing device 500 through blood opening 542 blood pressure rises, magnet 504 moves to the right, e.g., distally, against a restoring force provide by repulsion between moving magnet 504 and fixed magnet 505, instead of and/or in addition to a restoring force provided by a compressible fluid as described in association with the embodiments of FIGS. 1A-1C. Magnetic sensor 501 senses the movement of the magnet 504 through changes in the inductance of coil 510 relayed to a measurement unit via leads 515-1 and 515-2. The moving magnet 504 repels the fixed magnet 505 because like poles, such as north poles, face each other. As blood pressure decreases and blood is force out of the blood opening 542 by the repulsion force between magnet 504 and fixed magnet 505, magnetic sensor 501 senses the movement of the magnet 504 toward the magnetic sensor 501 through changes in the inductance of coil 510 which are relayed to a measurement unit via leads 515-1 and 515-2.

In one or more embodiments, a fixed magnet can be hollow, and a magnetic sensor is placed within a hole of the magnet. In such embodiments, the fixed magnet is at a proximal end of the sensing element and the moving magnet is at the distal end, therefore shortening the sensing element. If a saturable core magnetic sensor is used, the core material must have a saturation point H₀ that is approximately equal to the magnetic field within the hole of the fixed magnet.

FIG. 6 is a block diagram of an intravascular pressure sensing system in accordance with one or more embodiments of the present disclosure. In FIG. 6, a sensing device 660 is coupled to a measurement device 662. The sensing device 660 can be an intravascular magnetic pressure sensing device, such as the sensing device 100 described above in association with FIG. 1A. The sensing device 660 can be placed within a guidewire that is used in medical procedures that take place within a blood vessel.

In one or more embodiments, the measurement device 662 can include circuitry to receive, as an input, an electrical signal from the measurement device 662 and create an output based on the electrical signal from the measurement device 662. For example, the intravascular magnetic pressure sensing device can output an electrical signal through conductive sensing leads, e.g. leads 115-1 and 115-2 in FIG. 1A, to the measurement device 662. The measurement device 662 can receive the electrical signal as an input and using circuitry and/or a microprocessor to determine the impedance of windings around a sensing element. The impedance of the windings changes in response to a magnet moving due to pressure changes in the blood surrounding the sensing device 660. A look-up table can then be used by the measuring device 662 to determine a pressure that corresponds to the determined impedance.

Devices, systems, and methods associated with pressure sensing are described herein. In one or more embodiments, an intravascular pressure sensing device includes a magnetic sensing element fixedly positioned within a sensor tube, a magnet located a distance from the magnetic sensing element within the sensor tube, the magnet movably positioned within the sensor tube via a ferrofluid magnetically attached to the magnet, and an amount of compressible fluid sealed between the magnetic sensing element and the magnet.

It will be understood that, although the terms first, second, etc. may be used herein to describe various elements and that these elements should not be limited by these terms. These terms are only used to distinguish one element from another element. Thus, a first element could be termed a second element without departing from the teachings of the present disclosure.

Although specific embodiments have been illustrated and described herein, those of ordinary skill in the art will appreciate that an arrangement calculated to achieve the same results can be substituted for the specific embodiments shown. This disclosure is intended to cover adaptations or variations of various embodiments of the present disclosure.

It is to be understood that the above description has been made in an illustrative fashion, and not a restrictive one. Combination of the above embodiments, and other embodiments not specifically described herein will be apparent to those of skill in the art upon reviewing the above description. The scope of the various embodiments of the present disclosure includes other applications in which the above structures and methods are used. Therefore, the scope of various embodiments of the present disclosure should be determined with reference to the appended claims, along with the full range of equivalents to which such claims are entitled.

In the foregoing Detailed Description, various features are grouped together in a single embodiment for the purpose of streamlining the disclosure. This method of disclosure is not to be interpreted as reflecting an intention that the disclosed embodiments of the present disclosure have to use more features than are expressly recited in each claim.

Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. An intravascular pressure sensing device, comprising:
a magnetic sensing element fixedly positioned within a sensor tube;
a magnet located a distance from the magnetic sensing clement within the sensor tube, the magnet movably positioned within the sensor tube via a ferrofluid magnetically attached to the magnet; and
an amount of compressible fluid sealed between the magnetic sensing element and the magnet.

2. The device of claim 1, wherein the magnetic sensing element includes:
a wire filament wound around a non-hysteretic magnetic material; and
two conductive leads configured for providing signals to a measurement device.

3. The device of claim 2, wherein the non-hysteretic magnetic material is Metglas®.

4. The device of claim 1, wherein the compressible fluid sealed between the magnetic sensing element and the magnet includes an inert gas.

5. The device of claim 1, wherein the device is configured for incorporation into a pressure sensing guidewire.

6. The device of claim 1, wherein the magnetic sensing element is a Hall effect sensor.

7. The device of claim 1, wherein the magnetic sensing element is a giant magneto-resistive (GMR) sensor.

8. The device of claim 1, wherein a distal end of the sensor tube includes a stop member attached onto an inner surface of the sensor tube, the stop member configured to prevent movement of the magnet out of the sensor tube.

9. The device of claim 1, wherein the magnet is configured to move longitudinally within the sensor tube in response to changes in blood pressure with a body lumen.

10. An intravascular pressure sensing system, comprising:
a guidewire including an elongate tube and a core wire;
a first sensing device located within the elongate tube and including a first magnetic sensing element and a first movable magnet; and
a second sensing device located within the elongate tube and including a second magnetic sensing element and a second movable magnet.

11. The system of claim 10, wherein the first and second sensing devices are each positioned within a respective sensor tube.

12. The system of claim 11, wherein the first and second magnetic sensing elements are fixedly secured within the respective sensor tubes.

13. The system of claim 10, wherein at least one of the first and second magnetic sensors includes a saturable core sensor.

14. The system of claim 10, wherein at least one of the first and second magnets is at least partially surrounded by a ferrofluid.

15. The system of claim 10, wherein the guidewire includes a spring tip at a distal end, and wherein the first sensor tube is located proximal to the spring tip.

16. The system of claim 10, wherein the guidewire includes a proximal portion and a distal portion, and wherein the second sensor tube is located at a transition between the proximal portion and the distal portion.

17. The system of claim 10, wherein the first and the second sensing devices are spaced a distance apart such that the first sensing device is configured for measuring pressure distal to a coronary artery lesion and the second sensing device is configured for measuring pressure proximal to the coronary artery lesion.

18. The system of claim 10, wherein the first sensing element includes only two conductive leads which are coupled to and provide signals to a measurement device.

19. The system of claim 10, wherein the first sensing element includes only two conductive leads which are coupled to and provide signals to a measurement device and the second sensing element includes only two conductive leads, wherein the first sensing element and the second sensing element share a conductive lead, which are coupled to and provide signals to the measurement device.

20. An intravascular pressure sensing device, comprising:
a magnetic sensing element fixedly positioned within a sensor tube;
a first magnet located a distance from the magnetic sensing element within the sensor tube, the magnet movably positioned within the sensor tube via a ferrofluid magnetically attached to the magnet; and
a second fixedly positioned within a sensor tube,
wherein the first and second magnets are positioned such that a repulsive force exists between adjacent poles of the magnets.
